(19) United States
(12) Patent Application Publication
(10) Pub. No.: US 2019/0316292 A1
(43) Pub. Date: Oct. 17, 2019

(54) GRAPHENE THERMOSTATIC FABRICS AND METHODS OF MANUFACTURING THE SAME

(71) Applicant: Enerage Inc., Wujie Township (TW)

(72) Inventors: Mark Y. WU, Wujie Township (TW); Cheng-Yu HSIEH, Wujie Township (TW); Jing-Ru CHEN, Wujie Township (TW)

(21) Appl. No.: 16/291,292

(22) Filed: Mar. 4, 2019

(30) Foreign Application Priority Data
Apr. 13, 2018 (TW) 107112686

(51) Int. Cl. D06M 11/74; C01B 32/194; A41D 31/04; D06M 15/564; D06M 23/02; D06M 23/10

(52) U.S. Cl. CPC D06M 11/74; C01B 32/194; A41D 31/04; A61B 5/6804; D06M 23/02; D06M 23/10; D06M 15/564

(57) ABSTRACT

A graphene thermostatic fabric includes a fibrous tissue and a graphene thermostatic layer. The fibrous tissue has a first tissue surface, a second tissue surface and an interspace between the first tissue surface and the second tissue surface. The graphene thermostatic layer adheres to the first tissue surface, fills a part of the interspace, and includes at least a hydrophobic resin and nano-graphene sheets dispersed in the hydrophobic resin. A thermal conductivity of the graphene thermostatic layer varies with a change of an ambient temperature, and the thermal conductivity of the graphene thermostatic layer perpendicular to the first tissue surface is less than the thermal conductivity of the graphene thermostatic layer parallel to the first tissue surface. A method of manufacturing the graphene thermostatic fabric is further provided.

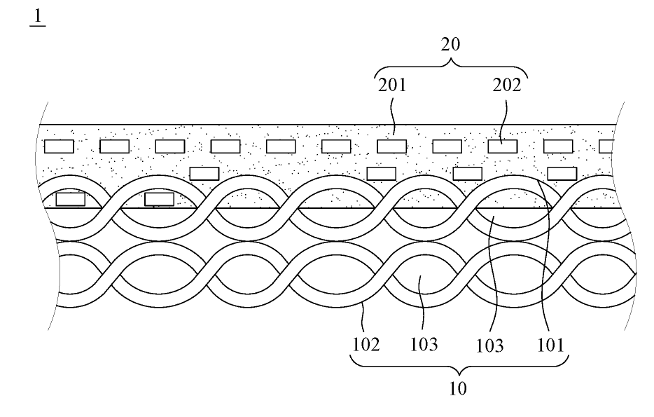

### CROSS-REFERENCE TO RELATED APPLICATION

This application claims the priority of Taiwanese patent application No. 107112686, filed on Apr. 13, 2018, which is incorporated herewith by reference.

### BACKGROUND OF THE INVENTION

### 1. Field of the Invention

The present application relates to a graphene thermostatic fabric having functions of heat preservation and dissipation, and a method of manufacturing the same.

### 2. The Prior Arts

With economic growth and increasing living levels, people have aesthetic requirements on textiles in addition to demand on basic use thereof. Recently, in response to extreme changes in the climate, the textiles that emphasize functionalities are popular; especially, cooling sensation clothes worn in hot weathers and warming sensation clothes worn in cold weathers have become daily clothes of ordinary people.

Current technologies that append the functions of cooling or warming sensation to the clothes mainly include adding functional materials related thereto in raw material of fibers, then spinning or drawing the raw material of fiber to produce functional fibers; or utilizing fabric tissue to achieve the functions. The patent application US 20170022634A1 discloses a technology for manufacturing composite yarns, the technology includes steps of uniformly mixing active particles (such as zeolite or activated carbon) and raw material of an artificial fiber, and then forming the composite yarns by wet spinning. A fabric made by the composite yarns has a property of fast moisture absorption, so as to produce the cooling effect.

However, an amount of the active particles added to the raw material would affect physical properties of the composite yarns, the higher amount the active particles is added, the worse strength the composite yarns have, the composite yarns having worse strength easily cause yarn breakage during the spinning process that affects a yarn yield; if a hardness of the active particles were too high, the active particles of high hardness would cause needle breakage during the weaving process that affects the fabric yield; on the other hand, if the amount of the active particles added therein were too low, the composite yarns and the fabric using the same could not have the physical properties required by the cooling effect.

Moreover, to achieve the cooling or warming effect requires adding different active particles in the fibers, there is no fabric in current market that can has both the cooling and warming effects. For example, the patent application CN 102747440 B discloses a technology of adding metal particles in fibers; or the patent application CN 104047368 B discloses a technology of adding aerogel in fibers, the fibers disclosed in the aforesaid patent applications only have the warming effect, but do not have the cooling effect.

Therefore, a primary aspect of the present application is to provide a fabric that has both the cooling and warming functions without affecting the yarn and fabric yield, and the fabric can pass the wash fastness test required by the industry.

### SUMMARY OF THE INVENTION

To achieve the aforesaid aspect, the present application provides a graphene thermostatic fabric including a fibrous tissue and a graphene thermostatic layer. The fibrous tissue has a first tissue surface, a second tissue surface and an interspace between the first tissue surface and the second tissue surface. The graphene thermostatic layer adheres to the first tissue surface and fills a part of the interspace, the graphene thermostatic layer includes at least a hydrophobic resin and nano-graphene sheets dispersed in the hydrophobic resin, wherein a thermal conductivity of the graphene thermostatic layer varies with a change of an ambient temperature, and the thermal conductivity of the graphene thermostatic layer perpendicular to the first tissue surface is less than the thermal conductivity of the graphene thermostatic layer parallel to the first tissue surface.

To achieve the aforesaid aspect, the present application provides a method of manufacturing the graphene thermostatic fabric including: mixing a first solvent and a second solvent to form a mixed solvent, wherein a boiling point of the first solvent is not greater than 80 Celsius degree (° C.), a boiling point of the second solvent is not less than 120° C., and a surface tension of the second solvent is between 30 and 60 mJ/m²; adding nano-graphene sheets to the mixed solvent, dispersing the nano-graphene sheets with a mechanical force to form a suspension solution of nano-graphene sheets; adding at least a hydrophobic resin to the suspension solution of the nano-graphene sheets, dispersing the nano-graphene sheets and the hydrophobic resin with the mechanical force, to form a graphene resin solution; and coating or printing the graphene resin solution on a surface of a fabric, removing the mixed solvent in the graphene resin solution, to form a graphene thermostatic layer adhering to the surface of the fabric.

The present application utilizes the graphene having the properties of anisotropic thermal conductivity, far infrared absorption and emission, and high conductivity, to manufacture the graphene thermostatic fabric; the method of manufacturing the graphene thermostatic fabric according to the present application includes steps of combining the solvents of low boiling and high surface tension to prepare the suspension solution of nano-graphene sheets, mixing the nano-graphene sheets and the hydrophobic resin to prepare the graphene resin solution, and coating or printing the graphene resin solution on the surface of fabric to allow the graphene thermostatic layer cover and embed a fabric tissue. When the ambient temperature is higher, the graphene thermostatic layer can accelerate heat dissipation from human skin to achieve the cooling effect; when the ambient temperature is lower, the graphene thermostatic layer can homogenize temperatures of different portions of the human skin, absorb infrared from the human skin, and then emit the far infrared to the human skin, so as to achieve the warming and thermostatic effects at the same time. In comparison with the current technologies of functional fibers, the graphene thermostatic fabric according to the present application has excellent adhesion and wash fastness; and the method of manufacturing the graphene thermostatic fabric according to the present application do not affect the fiber yield and efficiency of the drawing and weaving processes, effectively reduces a manufacturing cost; therefore, the graphene thermostatic fabric and the method of manufacturing the same according to the present application according to the present application have wide industrial utilization.

### BRIEF DESCRIPTION OF THE DRAWINGS

FIGURE schematically illustrates a cross-sectional view of a graphene thermostatic fabric according to the present application.

### DETAILED DESCRIPTION OF PREFERRED EMBODIMENTS

The technical features and other advantages of the present application will become more readily apparent to those ordinarily skilled in the art, by referring the following detailed description of embodiments of the present application in conjunction with the accompanying drawing. In order to further clarify the technical means adopted in the present application and the effects thereof, the FIGURE schematically illustrates the relative relationship between the main elements, but is not based on the actual size; therefore, thickness, size, shape, arrangement and configuration of the main elements in the FIGURE are only for reference, not intended to limit the scope of the present application.

The thermal conductivity of the graphene is higher than carbon nanotube and diamond, a resistance of the graphene is lower than copper or silver, and the graphene is the thinnest and hardness material as known in world. Recent researches find more unexpected physical properties of the graphene; for example, the thermal conductivity of the graphene varies the change of the ambient temperature, and a visible transmittance of the graphene is greater than 97% (i.e. a visible absorbance is less than 3%), but an infrared and microwave absorption rate of the graphene can reach 40%. Therefore, the present application utilizes the graphene having the properties of changeable thermal conductivity and higher infrared absorption rate, combines the graphene with specific resin to form the graphene thermostatic layer, and the graphene thermostatic fabric having both cooling and warming functions can be manufactured by using the graphene thermostatic layer. Additionally, due to the excellent conductivity of the graphene, the graphene thermostatic fabric according to the present application has an antistatic property, when a conductive material is further added to graphene thermostatic layer to act as a conductive line of a physiological sensor disposed on the functional clothes, design flexibility of the functional clothes can be effectively increased, and a manufacturing cost of the functional clothes can be greatly reduced.

FIGURE schematically illustrates a cross-sectional view of a graphene thermostatic fabric according to the present application. As shown in the FIGURE, a graphene thermostatic fabric 1 includes a fibrous tissue 10 and a graphene thermostatic layer 20. The fibrous tissue 10 has a first tissue surface 101, a second tissue surface 102, and an interspace 103 between the first tissue surface 101 and the second tissue surface 102. The graphene thermostatic layer 20 adheres to the first tissue surface 101, and fills a part of the interspace 103, the graphene thermostatic layer 20 includes at least a hydrophobic resin 201 and nano-graphene sheets 202 dispersed in the hydrophobic resin 201; and a thermal conductivity of the graphene thermostatic layer 20 varies with a change of an ambient temperature.

The fibrous tissue 10 is, for example but not limited to, plain weaved or knitted fabric of nylon, polyester, acrylic or the other fibers, a thickness of the fibrous tissue 10 usually is from 5 to 50 micrometer (μm). When the graphene thermostatic fabric is used as a clothes material, the first tissue surface 101 is an inner side facing human skin, the second tissue surface 102 is an outer side facing external environment, and sizes of the interspace 103 are inversely proportional to a number of fibers per unit area (i.e. fabric density), the higher the fabric density is, the less the air conducts heat between the human skin and the external environment through the interspace 103.

A thickness of the graphene thermostatic layer 20 is (for example, from 5 to 30 μm) not greater than the thickness of the fibrous tissue 10. The hydrophobic resin 201 is selected from polyurethane, polymethyl methacrylate, polyethylene terephthalate, and a combination thereof. The nano-graphene sheets 202 have a lamellar shape, a bulk density from 0.005 to 0.05 g/cm³, a thickness from 0.68 to 10 nm, and a lateral plane dimension from 1 to 100 μm.

A thermal conductivity of the hydrophobic resin 201 is far less the thermal conductivity of nano-graphene sheets 202, for example, the thermal conductivity of polyurethane is 0.02 W/mK. The nano-graphene sheets 202 is formed by a plurality of stacked graphene layers attracted to each other through van der Waals force, sp² covalent bond and honeycomb structure of the single graphene layer can rapidly conduct heat, but an out-of-plane (longitudinal) thermal conductivity along a thickness direction of the graphene layers is far less than an in-plane (lateral) thermal conductivity along the plane of the single graphene layer, a difference between the out-of-plane and in-plane thermal conductivities thereof at room temperature (25° C.) is above 10². The graphene thermostatic layer 20 formed by uniformly mixing the hydrophobic resin 201 and the nano-graphene sheets 202 has an anisotropic thermal conductivity far higher than the thermal conductivity of the hydrophobic resin.

The researches find that a theoretical thermal conductivity of the single graphene layer is changed by factors of lattice defects, impurities, lateral size, curling status and ambient temperature, a true mechanism that changes the thermal conductivity of the graphene is unclear; however, it has be confirmed that the thermal conductivity of the graphene sheets is substantially proportional to the change of the ambient temperature in a range below the absolute temperature 400 K, and the thermal conductivity of the graphene sheets is substantially inversely proportional to the change of the ambient temperature in a range above the absolute temperature 400 K.

In the graphene thermostatic layer 20 according to the present application, the nano-graphene sheets 202 account for 2 to 30 wt % of the graphene thermostatic layer 20. Through an actual test, the thermal conductivity of the graphene thermostatic layer 20 is not less than 0.8 W/mK at 30° C., the thermal conductivity thereof is not greater than 0.6 W/mK at 0° C. For example, when the ambient temperature is higher than 30° C., the greater thermal conductivity of the graphene thermostatic layer 20 contributes to body heat transfer and dissipation, so as to reduce a body temperature of an user; when the ambient temperature is lower than 0° C., the less thermal conductivity of the graphene thermostatic layer 20 can slow down the body heat dissipation. Usually, the temperature of human heart or back is higher, and the temperature of human body is lower; by utilizing the property that the lateral thermal conductivities of the graphene thermostatic layer is higher than the longitudinal thermal conductivity thereof, body heat can be transferred from the higher temperature portion to the lower temperature portion, so as to achieve the thermostatic or isothermal effect.

The other researches find that the far infrared (wavenumber from 33 to 333 cm⁻¹) absorbance of the graphene is up to 40%, and an infrared (wavenumber from 33 to 12800 cm⁻¹) radiance of the human skin is 98%, the range of radiance band (wavenumber) of the human skin overlaps the range of absorbance band of the graphene. When the ambient temperature is far lower than the temperature of the human skin to form a greater temperature difference, the graphene thermostatic layer 20 can absorb the infrared radiated from the human skin, then emit far infrared to the human skin to reduce heat loss caused by the greater temperature difference, so as to achieve the warming effect. Therefore, unlike most materials having only single function of heat dissipation or preservation, the graphene thermostatic layer 20 has both effects of heat dissipation and preservation at the same time.

It is worthy to say that the graphene has excellent conductivity, a volume resistance of the graphene thermostatic layer 20 including nano-graphene sheets 202 is from 10⁵ to 10¹² ohm*cm that can form a certain antistatic effect, the graphene thermostatic layer 20 can prevent the user's skin from harm of static electricity generated by the clothes in cold and dry environment, even the hydrophobic resin 201 of insulation is selected. When a conductive material (such as conductive carbon black) is further added to the graphene thermostatic layer 20 to reduce the volume resistance of the graphene thermostatic layer 20 to a range between 10¹ and 10⁵ ohm*cm, the graphene thermostatic layer 20 can have conductivity. A patterned graphene thermostatic layer 20 that partially cover the first tissue surface 101 is formed with printing, a physiological sensor (not shown) for detecting human physiological signal is disposed on the first tissue surface 101 and connects to the patterned graphene thermostatic layer 20, the patterned graphene thermostatic layer 20 having conductivity can be used as a signal transmission line of the physiological sensor, so that the graphene thermostatic fabric 1 can be utilized in medical monitoring field.

The present application provides a method of manufacturing the graphene thermostatic fabric, the method includes following steps. A step of preparing a solvent, a first solvent and a second solvent are mixed to form a mixed solvent, wherein a boiling point of the first solvent is not greater than 80° C., and a boiling point of the second solvent is not less than 120° C. A step of preparing a suspension solution of nano-graphene sheets, the nano-graphene sheets are added to the mixed solvent, and dispersed with a mechanical force, to form the suspension solution of the nano-graphene sheets. A step of preparing a graphene resin solution, at least a hydrophobic resin is added to the suspension of the nano-graphene sheets, and the nano-graphene sheets and the hydrophobic resin are dispersed with the mechanical force, to form the graphene resin solution. A step of forming a graphene thermostatic layer, the graphene resin solution is coated or printed over a surface of a fabric, and the mixed solvent in the graphene resin solution is removed, to form the graphene thermostatic layer adhering to the surface of the fabric.

In the step of preparing the solvent, due to a surface tension of the graphene is about 45-50 mJ/m², if a difference of the surface tensions between the graphene and the solvents were too great, the nano-graphene sheets would be easily agglomerated and precipitated, and not easily dispersed; the solvent having the surface tension close to the graphene contributes to the dispersion of the graphene, but is not easily removed; therefore, the first solvent of lower boiling point and the second solvent of surface tension close to the graphene are combined to form the mixed solvent for preparing the suspension solution of the nano-graphene sheets. Specifically, the first solvent is selected from acetone, butanone, ethyl acetate, butyl acetate, and a combination thereof; the second solvent is selected from N,N-dimethyl acetamide, dimethyl sulfoxide, dimethyformamide, dimethylacetamide, and a combination thereof.

In the step of preparing the suspension solution of the nano-graphene sheets, due the surface tensions of second solvent and the graphene are about matched, the nano-graphene sheets can be effectively dispersed in the mixed solvent with the mechanical force (for example, ultrasonic, homogenous agitation, ball milling, and high pressure shearing) of general dispersion equipment.

In the step of preparing the graphene resin solution, due the mixed solvent can keep the dispersion of the nano-graphene sheets, even the hydrophobic resin has high viscosity, the mechanical force of aforesaid dispersion equipment is sufficient to uniformly disperse the nano-graphene sheets in the graphene resin solution. Specifically, the hydrophobic resin is selected from polyurethane, polymethyl methacrylate, polyethylene terephthalate, and a combination thereof.

In the step of forming the graphene thermostatic layer, there are not particular restrictions on types of the fabric (for example, knitted fabrics), the fibrous tissue of the fabric has an interspace; the graphene resin solution completely covers the surface of the fabric with blade coating, or partially covers the surface of the fabric with screen printing; the mixed solvent is removed by heating the graphene resin solution, and the (patterned) graphene thermostatic layer, which completely (or partially) covers the surface of the fabric and embeds the interspace of the fibrous tissue, is formed.

In order that the specific effects and advantages of the graphene thermostatic fabric will be more apparent to those skilled in the art, the graphene thermostatic fabric will be described in details with following exemplary embodiments according to the present application.

### Exemplary Embodiment 1

A butanone is used as the first solvent, a dimethylacetamide is used as the second solvent, and the butanone and the dimethylacetamide are mixed in a volume ration of 8:2, to form the mixed solvent. The nano-graphene sheets are added to the mixed solvent in a weight ratio of 10:90, and the nano-graphene sheets are uniformly dispersed in mixed solvent by using a homogenizer, to form the suspension solution of the nano-graphene sheets (i.e. the nano-graphene sheets account for 10 wt % of the suspension of the nano-graphene sheets). A polyurethane resin of 900 g is added to the suspension solution of the nano-graphene sheets of 1000 g, and the nano-graphene sheets and the polyurethane resin are dispersed by using the homogenizer, to form the graphene resin solution. The graphene resin solution is printed over a tissue surface of the knitted fabric with gravure printing, the mixed solvent is removed by heating the graphene resin solution to 100° C., and the graphene thermostatic fabric having the graphene thermostatic layer is formed.

### Exemplary Embodiment 2

A butanone is used as the first solvent, a dimethyl sulfoxide is used as the second solvent, and the butanone and the dimethyl sulfoxide are mixed in a volume ratio of 9:1, to form the mixed solvent. The nano-graphene sheets are added to the mixed solvent in a weight ratio of 15:85, and the nano-graphene sheets are uniformly dispersed in the mixed solvent by using the homogenizer, to form the suspension solution of the nano-graphene sheets (i.e. the nano-graphene sheets account for 15 wt % of the suspension solution of the nano-graphene sheets). A polyurethane resin of 800 g is added to the suspension solution of the nano-graphene sheets of 300 g, the nano-graphene sheets and the polyurethane resin are dispersed by using a revolutionary rotation mixer at a rotation speed of 1000 rpm and a revolution speed of 400 rpm, to form the graphene resin solution. The graphene resin solution is printed over a tissue surface of the knitted fabric with screen printing, the mixed solvent is removed by heating the graphene resin solution to 100° C., and the graphene thermostatic fabric having the graphene thermostatic layer is formed.

### Exemplary Embodiment 3

A butanone is used as the first solvent, a dimethyl sulfoxide is used as the second solvent, and the butanone and the dimethyl sulfoxide are mixed in a volume ratio of 9:1, to form the mixed solvent. The nano-graphene sheets are added to the mixed solvent in a weight ratio of 15:85, and the nano-graphene sheets are uniformly dispersed in the mixed solvent by using the homogenizer, to form the suspension solution of the nano-graphene sheets (i.e. the nano-graphene sheets account for 15 wt % of the suspension solution of the nano-graphene sheets). A polyurethane resin of 800 g is added to the suspension solution of the nano-graphene sheets of 400 g, the nano-graphene sheets and the polyurethane resin are dispersed by using the revolutionary rotation mixer at the rotation speed of 1000 rpm and the revolution speed of 400 rpm, to form the graphene resin solution. The graphene resin solution is coated over a surface of a release substrate (for example, polyester film) with blade coating, the mixed solvent is removed by heating the graphene resin solution to 100° C., to form the graphene thermostatic layer coated on the release film. The graphene thermostatic layer coated on the release film and a knitted fabric are heated and pressed, then the release film is removed, and the graphene thermostatic fabric is formed.

### Thermal Conduction Test

The thermal conductivities of the graphene thermostatic fabrics of Exemplary embodiments 1 to 3 are measured according to the testing standard of ASTM D7984. The test results are shown in Table 1.

TABLE 1

| | | | A difference |
|---|---|---|---|
| | | | between the |
| | Thermal | Thermal | thermal |
| | conductivity | conductivity | conductivities |
| | (W/mK) | (W/mK) | at 273 K and |
| Testing sample | at 273 K | at 303 K | 303 K (%) |
| Exemplary embodiment 1 | 0.58 | 0.82 | 29.2% |
| Exemplary embodiment 2 | 0.58 | 0.85 | 31.7% |
| Exemplary embodiment 3 | 0.59 | 0.86 | 31.4% |

As shown in Table 1, with increasing the added amount of the nano-graphene sheets, the thermal conductivity of the graphene thermostatic fabric is increased, and the effect of conducting heat thereof is better.

### Far Infrared Test

The graphene thermostatic fabric of Exemplary embodiment 3 is measured by using an infrared spectrophotometer, and a stable emissivity of the graphene thermostatic fabric is 0.9 in a wavelength range from 2 to 22 μm. In comparison with a knitted fabric without the graphene thermostatic layer, temperature rise of the graphene thermostatic fabric is 0.8° C. higher. It can be seen that the properties of the graphene thermostatic fabric absorbing and emitting far infrared produce the heat preservation effect.

### Static Temperature Rise Test

The graphene thermostatic fabric of Exemplary embodiment 3 and a knitted fabric without graphene thermostatic layer are respectively irradiated with a halogen lamp of 500 W for 10 minutes, and then the temperature rises of the two fabrics are observed with an infrared camera. The temperature rise of the graphene thermostatic fabric is 2° C. higher than the temperature rise of the knitted fabric. It shows that the graphene thermostatic fabric can accelerate the temperature rise of the fabrics, and enhance the heat preservation effect.

### Instant Cooling Test

Instant heat dissipations of the graphene thermostatic fabrics of Exemplary embodiments 1 to 3 are measured according to testing method of FTTS-FA-019 (Q-max). The measurements are shown in Table 2.

TABLE 2

| Testing sample | Instant heat flux of the fabrics (W/cm²) |
|---|---|
| Exemplary embodiment 1 | 1.030 |
| Exemplary embodiment 2 | 1.060 |
| Exemplary embodiment 3 | 1.637 |

As shown in Table 2, the instant heat flux of graphene thermostatic fabrics are far higher than the testing standard of cooling fabrics (0.14 W/cm²), and the instant heat flux of the graphene thermostatic fabrics is increased with increasing the amount of the nano-graphene sheets added therein.

### Exemplary Embodiment 4

A butanone is used as the first solvent, a dimethyl sulfoxide is used as the second solvent, and the butanone and the dimethyl sulfoxide are mixed in a volume ratio of 9:1, to form the mixed solvent. The nano-graphene sheets and natural graphite powder are mixed in a weight ratio of 1:2, the nano-graphene sheets mixed with the natural graphite powder are added to the mixed solvent in a weight ratio of 20:80, and the nano-graphene sheets mixed with the natural graphite powder are uniformly dispersed in the mixed solvent by using the homogenizer, to form the suspension solution of the nano-graphene sheets (i.e. the nano-graphene sheets mixed with the natural graphite powder account for 20 wt % of the suspension solution of the nano-graphene sheets). A polyurethane resin of 800 g is added to the suspension solution of the nano-graphene sheets of 120 g, the nano-graphene sheets, the natural graphite powder and the polyurethane resin are dispersed by using the revolutionary rotation mixer at the rotation speed of 1000 rpm and the revolution speed of 400 rpm, to form the graphene resin solution. The graphene resin solution is coated over a surface of a release substrate (for example, polyester film) with the blade coating, the mixed solvent is removed by heating the graphene resin solution to 100° C., to form the graphene thermostatic layer coated on the release film. The graphene thermostatic layer coated on the release film and a knitted fabric are heated and pressed, then the release film is removed, and an antistatic graphene thermostatic fabric having a surface resistance of 3*10⁷ ohm/sq is formed.

### Exemplary Embodiment 5

A butanone is used as the first solvent, a dimethyl sulfoxide is used as the second solvent, and the butanone and the dimethyl sulfoxide are mixed in a volume ratio of 9:1, to form the mixed solvent. The nano-graphene sheets and conductive carbon black are mixed in a weight ratio of 1:3, the nano-graphene sheets mixed with the conductive carbon black are added to the mixed solvent in a weight ratio of 23:77, and the nano-graphene sheets mixed with the conductive carbon black are uniformly dispersed in the mixed solvent by using a high pressure homogenizer, to form the suspension solution of the nano-graphene sheets (i.e. the nano-graphene sheets mixed with the conductive carbon black account for 23 wt % of the suspension solution of the nano-graphene sheets). A polyurethane resin of 800 g is added to the suspension solution of the nano-graphene sheets of 115 g, the nano-graphene sheets, the conductive carbon black and the polyurethane resin are dispersed by using the revolutionary rotation mixer at the rotation speed of 1000 rpm and the revolution speed of 400 rpm, to form the graphene resin solution. The graphene resin solution is coated over a surface of a release substrate (for example, polyester film) with the blade coating, the mixed solvent is removed by heating the graphene resin solution to 100° C., to form the graphene thermostatic layer coated on the release film. The graphene thermostatic layer coated on the release film and a knitted fabric are heated and pressed, then the release film is removed, and the antistatic graphene thermostatic fabric having a surface resistance of 2*10⁵ ohm/sq is formed.

### Exemplary Embodiment 6

The nano-graphene sheets of 40 g, a carbon black of 40 g and an isophorone of 400 g are mixed by using the homogenizer, to form the suspension solution of the nano-graphene sheets; the suspension solution of the nano-graphene sheets of 480 g and a polyester resin of 230 g are mixed by using the revolutionary rotation mixer at the rotation speed of 1000 rpm and the revolution speed of 400 rpm, to form the graphene resin solution having a viscosity greater than 20,000 cps; the graphene resin solution is added in a dispersing equipment, the nano-graphene sheets and the carbon black are uniformly dispersed in the polyester resin through a first dispersing process and a second dispersing process of the dispersing equipment, wherein the first dispersing process includes setting a pressure at 20 bar and a slit at 150 μm, and allowing the graphene resin solution pass through the slit at a flow rate of 1 L/min, the second dispersing process includes setting the pressure at 24 bar and the slit at 30 μm, and allowing the graphene resin solution pass through the slit at a flow rate of 2.0 L/min; the graphene resin solution is printed over a surface of a knitted fabric by using a screen of 200-mesh; the mixed solvent in the graphene resin solution is removed by heating knitted fabric at 130° C., to form the graphene thermostatic fabric having a conductive graphene thermostatic layer. The graphene thermostatic fabric of Exemplary embodiment 6 has a surface resistance of 210 ohm/sq that meets a requirement of a conductive line of a physiological sensor.

### Wash Fastness Test

The graphene thermostatic fabrics of Exemplary embodiments 4 to 6 are washed with water for 20 times according to testing standard of AATCC 135, the surface resistances of the graphene thermostatic fabrics before and after the washing are measured to test adhesion fastness of the graphene thermostatic layers. The measurements are shown in Table 3.

TABLE 3

| | Measurement of surface resistance (ohm/sq) | |
|---|---|---|
| Testing sample | before the washing | after the washing |
| Exemplary embodiment 4 | 2*10⁷ | 5*10⁷ |
| Exemplary embodiment 5 | 3*10⁵ | 5*10⁵ |
| Exemplary embodiment 6 | 210 | 265 |

As shown in Table 3, even the natural graphite powder or the conductive carbon black are added in the graphene thermostatic fabrics, the surface resistances of the graphene thermostatic fabrics do not have much difference before and after the washing; especially, the surface resistance of the graphene thermostatic layer of Exemplary embodiment 6 after the washing still meets the resistance requirement of the conductive line of the physiological sensor that can prove the graphene thermostatic fabric according to the present application has excellent adhesion and wash fitness.

In summary, the present application utilizes the graphene having the special thermal properties and high conductivity to manufacture the graphene thermostatic fabric; the method of manufacturing the graphene thermostatic fabric according to the present application includes steps of combining the solvents of low boiling and high surface tension to prepare the suspension solution of nano-graphene sheets, mixing the nano-graphene sheets and the hydrophobic resin to prepare the graphene resin solution, and coating or printing the graphene resin solution on the surface of fabric to allow the graphene thermostatic layer cover and embed a fabric tissue. When the ambient temperature is higher, the graphene thermostatic layer can accelerate the heat dissipation from the human skin to achieve the cooling effect; when the ambient temperature is lower, the graphene thermostatic layer can homogenize the temperatures of different portions of the human skin, absorb infrared from the human skin, and then emit the far infrared to the human skin, so as to achieve the warming and thermostatic effects at the same time. In comparison with the current technologies of functional fibers, the graphene thermostatic fabric according to the present application has excellent adhesion and wash fastness; and the method of manufacturing the graphene thermostatic fabric according to the present application do not affect the fiber yield and efficiency of the drawing and weaving processes, effectively reduces a manufacturing cost; therefore, the graphene thermostatic fabric and the method of manufacturing the same according to the present application have wide industrial utilization.

The exemplary embodiments described above only illustrate the principles and effects of the present application, but are not intended to limit the scope of the present application. Based on the above description, an ordinarily skilled in the art can complete various similar modifications and arrangements according to the technical programs and ideas of the present application, and the scope of the appended claims of the present application should encompass all such modifications and arrangements.

What is claimed is:

1. A graphene thermostatic fabric, comprising:
a fibrous tissue, having a first tissue surface, a second tissue surface and an interspace between the first tissue surface and the second tissue surface; and
a graphene thermostatic layer, adhering to the first tissue surface and filling a part of the interspace, comprising at least a hydrophobic resin and nano-graphene sheets dispersed in the hydrophobic resin, wherein a thermal conductivity of graphene thermostatic layer varies with a change of an ambient temperature, and the thermal conductivity of the graphene thermostatic layer perpendicular to the first tissue surface is less than the thermal conductivity of the graphene thermostatic layer parallel to the first tissue surface.

2. The graphene thermostatic fabric according to claim 1, wherein the hydrophobic resin is selected from polyurethane, polymethyl methacrylate, polyethylene terephthalate, and a combination thereof.

3. The graphene thermostatic fabric according to claim 1, wherein the nano-graphene sheets has a bulk density from 0.005 to 0.05 g/cm³, a thickness from 0.68 to 10 nm, and a lateral plane dimension from 1 to 100 micro-meter (μm).

4. The graphene thermostatic fabric according to claim 1, wherein the nano-graphene sheets account for 2 to 30 wt % of the graphene thermostatic layer.

5. The graphene thermostatic fabric according to claim 1, wherein a thickness of the graphene thermostatic layer is between 5 and 30 μm.

6. The graphene thermostatic fabric according to claim 1, wherein the graphene thermostatic layer completely covers the first tissue surface.

7. The graphene thermostatic fabric according to claim 1, wherein the graphene thermostatic layer is patterned, and partially covers the first tissue surface.

8. The graphene thermostatic fabric according to claim 1, when the ambient temperature is not greater than 400 K, the thermal conductivity of the graphene thermostatic layer is proportional to the change of the ambient temperature.

9. The graphene thermostatic fabric according to claim 8, when the ambient temperature is at 303 K, the thermal conductivity of the graphene thermostatic layer is not less than 0.8 W/mK.

10. The graphene thermostatic fabric according to claim 8, when the ambient temperature is at 273 K, the thermal conductivity of the graphene thermostatic layer is not greater than 0.6 W/mK.

11. The graphene thermostatic fabric according to claim 1, wherein a volume resistance of the graphene thermostatic layer is between 10⁵ and 10¹² ohm*cm.

12. The graphene thermostatic fabric according to claim 1, wherein the graphene thermostatic layer further comprises: conductive carbon black, graphite, or a combination thereof, a volume resistance of the graphene thermostatic layer is between 10¹ and 10⁵ ohm*cm.

13. The graphene thermostatic fabric according to claim 12, further comprising a physiological sensor disposed on the first tissue surface, wherein the graphene thermostatic layer is used for transmitting an electronic signal produced by the physiological sensor.

14. A method of manufacturing a graphene thermostatic fabric, comprising:
mixing a first solvent and a second solvent to form a mixed solvent, wherein a boiling point of the first solvent is not greater than 80 Celsius degree, a boiling point of the second solvent is not less than 120 Celsius degree, and a surface tension of the second solvent is between 30 and 60 mJ/m²;
adding the nano-graphene sheets to the mixed solvent, dispersing the nano-graphene sheets with mechanical force to form a suspension solution of the nano-graphene sheets;
adding at least a hydrophobic resin to the nano-graphene sheets suspension solution, dispersing the nano-graphene sheets and the hydrophobic resin with the mechanical force, to form a graphene resin solution; and
coating or printing the graphene resin solution on a surface of a fabric, removing the mixed solvent in the graphene resin solution, to form a graphene thermostatic layer adhering to the surface of the fabric.

15. The method of manufacturing the graphene thermostatic fabric according to claim 14, wherein the first solvent is selected from acetone, butanone, ethyl acetate, butyl acetate, and a combination thereof.

16. The method of manufacturing the graphene thermostatic fabric according to claim 14, wherein the second solvent is selected from N,N-dimethylacetamide, dimethyl sulfoxide, dimethyformamide, dimethylacetamide, and a combination thereof.

17. The method of manufacturing the graphene thermostatic fabric according to claim 14, wherein the mechanical force is selected from ultrasonic, homogenous agitation, ball milling, and high pressure shearing.

18. The method of manufacturing the graphene thermostatic fabric according to claim 14, wherein the nano-graphene sheets account for 4 to 40 wt % of the graphene suspension solution.

19. The method of manufacturing the graphene thermostatic fabric according to claim 14, wherein the hydrophobic resin is selected from polyurethane, polymethyl methacrylate, polyethylene terephthalate, and a combination thereof, and the nano-graphene sheets account for 2 to 30 wt % of the graphene thermostatic layer.

20. The method of manufacturing the graphene thermostatic fabric according to claim 14, further comprising:
adding a conductive material to the nano-graphene sheets suspension solution, to form the graphene resin solution; and
disposing at least a physiological sensor on the surface of the fabric, and the graphene thermostatic layer connects to the physiological sensor.
